(19) United States
(12) Patent Application Publication
(10) Pub. No.: US 2011/0054366 A1
(43) Pub. Date: Mar. 3, 2011

(54) THERAPEUTIC PRESSURE SYSTEM

(76) Inventors: Kent Smith, San Diego, CA (US); Mark McWilliams, San Diego, CA (US); William Bloom, Encinitas, CA (US); Mark P. Banister, Tucson, AZ (US)

(21) Appl. No.: 12/918,466

(22) PCT Filed: Feb. 19, 2009
(86) PCT No.: PCT/US09/34557

§ 371 Date: Nov. 8, 2010

Related U.S. Application Data
(60) Provisional application No. 61/029,818, filed on Feb. 19, 2008.

(51) Int. Cl. A61H 7/00; A61F 7/08; A61F 7/10

(52) U.S. Cl. 601/15; 601/84

(57) ABSTRACT

A therapeutic pressure system includes a sealed actuator housing containing an electrolyte and one or more polymer actuators that actuate via movement of electrolyte components into, out of around or within the polymer matrix of the polymer actuators. The system also includes an actuator support structure for the polymer actuators, working and counter electrodes in working contact with the actuators, circuitry for connecting the electrodes to a power source, and a controller between the power source and the electrodes to control the electrical current to the electrodes. The system is particularly useful for use in a hospital bed.

This invention relates to the field of pressure relief systems to apply or relieve pressure and providing comfort to a patient. The invention has particular utility as described in connection with hospital bed pads or mattresses and will be described in connection with such utility, although other utilities including boots, socks or cuffs for preventing deep vein thrombosis (DVTs) to be worn by a patient during a surgery, after surgery, as well as sitting chair and wheelchair seat pads, and pressure wraps for the body or extremities are contemplated.

Pressure relief for patients confined to beds for significant periods of time is a significant problem. Pressure sores, such as decubitus ulcers, potentially lead to infections and other conditions or complications. These can occur from prolonged pressure exposure, such as experienced by those confined to beds, whether in a hospital, nursing home, or private residence. Considerable efforts have been made to provide mattress systems or patient support surfaces which effectively redistribute and/or equalize pressure forces at the interactions between the patient and the support surface. Generally speaking, the more sophisticated systems for achieving such pressure reductions are expensive to manufacture and maintain. Today, several vendors manufacture support surfaces which include various elongated air tubes, cells or cylinders combined with foam pieces or perimeters which surround the air tubes. Examples of embodiments having multiple, elongated air tubes are set forth in U.S. Pat. Nos. 5,692,256; 5,412,821, and 5,070,560. These air tubes or bladders are somewhat large and cannot change the pressure over small areas or locations of the patient's skin within an area where pressure is already applied. Another deficiency of prior art systems is that air or other gasses are highly compressible and thereby not able to apply an accurate amount of force to a specific location within an area in contact with the patient without changing or moving a large surface area in contact with the patient.

Consider, for example, the bed shown in U.S. Pat. No. 5,692,256 which is similar to one available commercially from Hill-Rom, and which requires a special stepped deck which includes an upper deck, and a lower deck coupled to the upper deck by a vertical deck side wall. Recently, the manufacturer has angled the side wall of its bed having stepped decks so that the space between the outermost air cell and the side wall is occupied with a resilient foam bolster. These bolsters, however, limit the amount of area occupied by the air cells.

Hospital beds such as above described are expensive to manufacture and maintain, and have a tendency, due to their design, to be hot, transferring heat to a patient's body, creating an uncomfortable condition for the patient. Also, since current pressure relief systems such as above described require specialized bed frames, they cannot be used with conventional hospital mattresses.

In view of the foregoing deficiencies associated with conventional support surfaces and beds, there remains a need for a support surface which can respond to the needs of the patient by activating small areas of the usable surface area creating a pressure changing surface that will not disturb or move the patient.

In co-pending U.S. published application 2004/0234401 and PCT application numbers US2004/05922, US2005/45210 and US2007/73188, all assigned to a common assignee, and incorporated herein by reference, there are described unique polymer actuators for transferring fluids. While described primarily for use in connection with infusion pumps for delivering drugs to patients, we have found that, with modification these polymer actuators advantageously may be used as a motive source for therapeutic pressure systems.

More particularly, the present invention in one aspect, provides a therapeutic pressure system comprising one or more sealed actuator housings containing an electrolyte, one or more polymer actuators that actuate via movement of electrolyte components into, out of, around or within the polymer matrix of the polymer actuators, an actuator support structure to hold the polymer actuators, working and counter electrodes in working contact with the actuators, circuitry for connecting the electrodes to a power source, and a controller to control electrical current to the electrodes.

The actuators can be compressible or non compressible thereby providing a range of high or low pressure needed over small areas, without the associated discomfort of pressure over large areas such as normally experienced with conventional therapeutic pressure systems. The therapeutic pressure system is made to be programmable and reactive to the needs of the patient using the system and is made from a combination of materials depending on the specific use, therapies and patients needs, for example, concerning patient support surfaces, and, if desired, may be combined with foam and air bladder technology used in current therapeutic bedding and the like. Pressure distribution across a patients body surface can be controlled in such a manner as to reduce pressure points which would exceed capillary pressure leading to a cessasion of blood flow into sub-surface tissues and can be further controlled to create pressure waves in a manner that moves sub-surface interstitial fluids, both enabling and promoting blood flow to these tissues. Also, the actuators of the present invention may be programmed to move in sequence or alternating pressure waves thereby facilitating blood flow deep within the patient to prevent not only problems that arise from deep vein thrombosis (DVT's), and also other blood flow complications such as bed sores or ulcers caused by restricted blood flow at the capillary surface or skin level of the patient.

In a preferred embodiment of the invention, the therapeutic pressure system includes temperature and/or pressure sensors and controllers which permit control of the polymer actuators responsive to temperatures and pressures.

The therapeutic pressure system of the present invention may be encased within bedding materials such as polymer foams or padding which form a support surface for the patient, and which include a foam or padded perimeter, a shaped core, a topper, and a coverlet. The foam or padded perimeter surrounds an interior well, defined by a floor, a top surface, and spaced apart side walls. The core may be comprised of a plurality of geometrically shaped cells. Each cell has a bottom wall, a top wall, and one or more side walls and may be structured as an enclosed system.

One aspect of the present invention provides a modular construction for a grouping of different patient support surfaces involving combinations of foam, polymer actuators and air which are facilitated by modular assembly components. Another aspect provides a modular actuator system which may be used in common with a number of different embodiments of patient support surfaces manufactured by various vendors.

In yet another aspect of the present invention there are provided inner bolster and foam topper constructions which work in concert with integrated polymer actuators or cylinders. Preferably, the invention includes interlocking, integrated designs which provide flexible, progressive support while maximizing structural integrity of the patient support system.

Another aspect of the present invention is to provide improved patient support surfaces with reference to patient safety. Additionally, it should provide improved patient protection against unintended rolling near the edge of the patient support surface or unintended forward pitching from the edge of the support surface during entry. It is also a feature of the invention to facilitate safer transfers and more stable edge-of-bed sitting. In such context, it is intended to provide patient support surfaces which gently prompt a patient towards the desired area of the bed without requiring awkward buildups or structures which would otherwise obstruct entry to or egress from such patient support surfaces.

Another aspect of the invention is to provide improved foot comfort by redistributing and equalizing loads to the more relatively pressure-tolerant lower legs and calves. It is a specific goal to achieve such improved foot comfort and improved patient health by providing particular foot pressure relief sections incorporated into various embodiments of foam or other mattress toppers integrally built into different types of patient support surfaces. Yet other embodiments may include foam toppers which have integrally incorporated a sloping lower leg/foot pressure relief section for improved patient foot health in accordance with this invention.

One embodiment of the present invention relates to improved patient support surfaces having perimeter bolster features which facilitate structural integrity and patient protection. Other embodiments include combinations of inner bolster features and foam toppers with underside features for engaging and capturing prepositioned longitudinal and/or lateral placed polymer actuators, for improved patient support surface integrity and performance.

Still, other embodiments include various combinations of the foregoing features so as to result in a modular assembly common to different embodiments of static or dynamic and/or non-powered or powered patient support surface constructions. Further preferred embodiments relate to the methodology involved with such mattress features which comprise static non-powered air cylinder configuration combined with polymer actuated configurations.

Still other advantages of the invention are the incorporation of sensors within the actuator system for monitoring of the patient or monitoring the therapy effects and then giving ‘real time’ feedback to the controller so it automatically can adjust the motion of the actuators or the temperature, etc. needed to provide maximum comfort and therapeutic effect for the patient. The controller can connect to a central monitoring system either wirelessly or by wire so that multiple patients can be monitored at a time thereby providing a more efficient tool for the nursing or care provider staff.

Additional features and advantages of the invention are obvious or will be apparent to those of ordinary skill in the art of the applicable use or therapy. Also, it should be further appreciated that modifications and variations to the specifically illustrated and discussed features and steps or materials and devices may be practiced in various embodiments and uses of this invention without departing from the spirit and scope thereof, by virtue of present reference thereto. Such variations may include, but are not limited to, substitution of equivalent means and features, materials, or steps for those shown or discussed, and the functional or positional reversal of various parts, features, steps, or the like.

Further features and advantages of the present invention will be seen taken in conjunction with the accompanying drawings, wherein like numerals depict like parts, and wherein

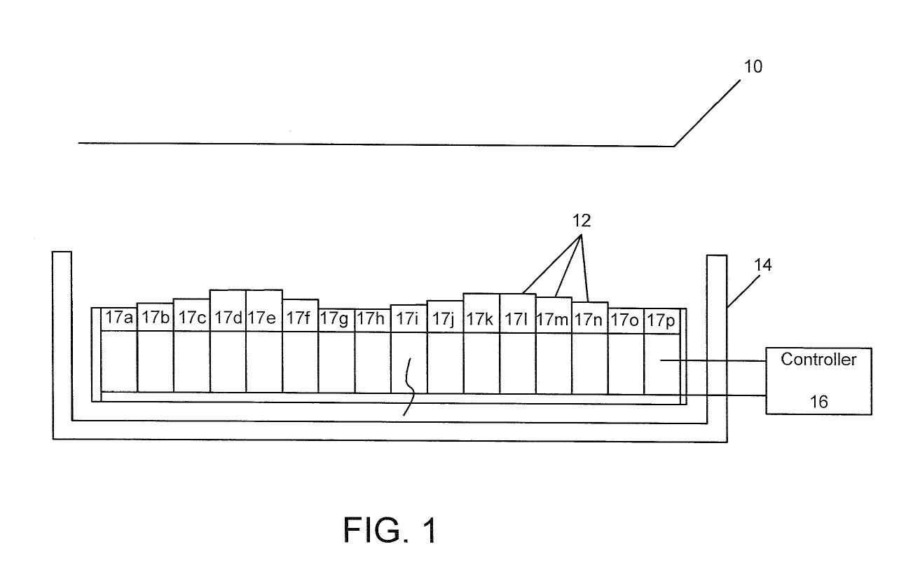

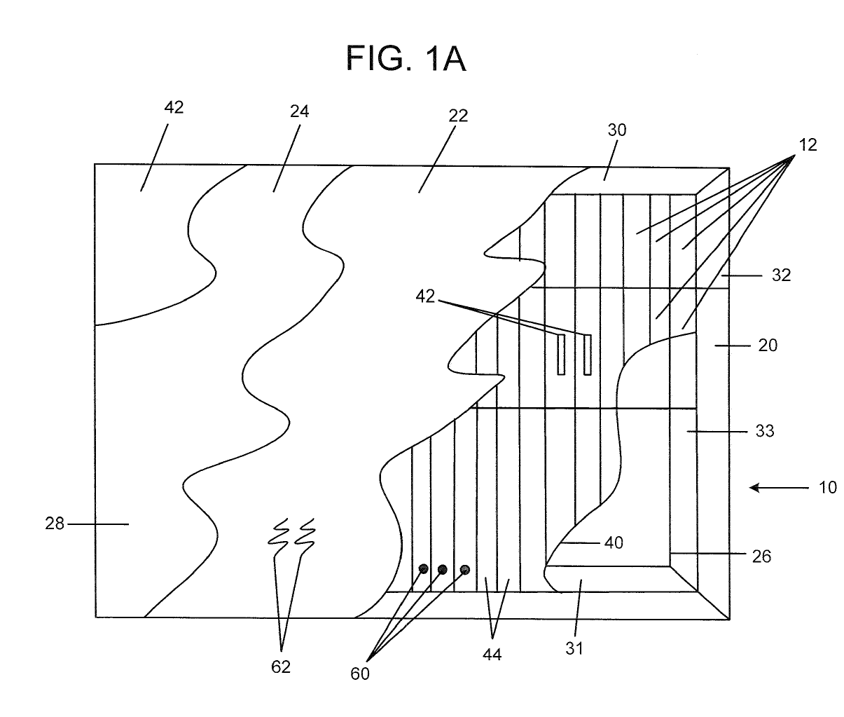

FIG. 1 is a sectional view of a patient's support system in a hospital bed in accordance with one embodiment of the invention;

FIG. 1A is a top plan view, in partial cross-section of the patient support system of FIG. 1;

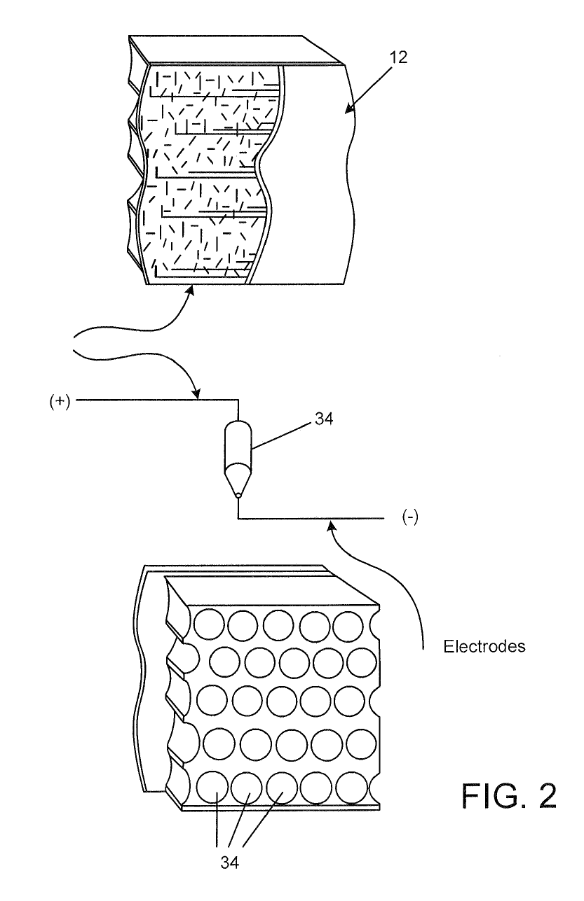

FIG. 2 is a schematic of a pressure actuator and control in accordance with the present invention;

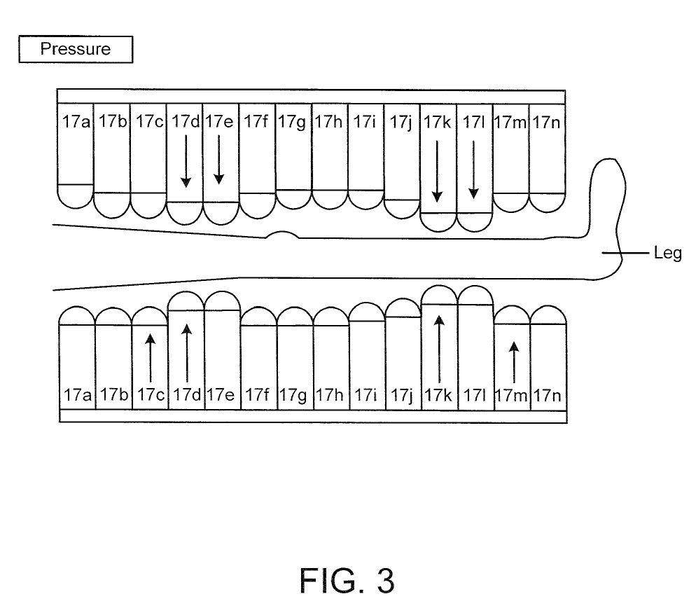

FIG. 3 is a sectional view of a second embodiment of the invention in the form of a patient pressure boot; and

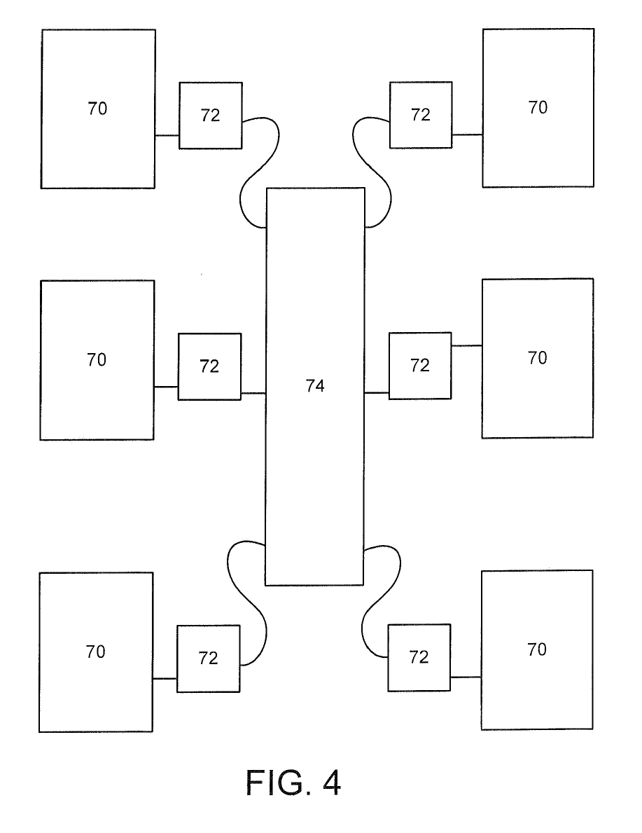

FIG. 4 is a schematic view showing a plurality of patient support systems communicating with a central monitoring system.

Referring to the drawings, there is shown in FIGS. 1 and 1A, a patient support system 10 in an embodiment of the invention. The patient support system 10 includes a plurality of polymer actuator assemblies 12 shaped in form of a mattress, a housing 14 for holding the actuator assemblies 12, and controller 16 as will be described in detail below. The actuator assembly 12 and its housing 14 are adapted to be coupled with a bed 18 similar to that shown in U.S. Pat. No. 5,692,256, which is incorporated herein by reference.

Referring also to FIG. 1A, the actuator assemblies 12 and their housing 14 are in the form of a generally geometrically shaped core and have a foam perimeter 20, a topper 22, and a coverlet 24. The topper 22 may be made of any conventionally known mattress topper material, such as resilient polyurethane, or one or more layers of different padding materials.

The coverlet 24 is configured to overlay the topper 22 and the top portion of the perimeter. The coverlet 24 may be made of any conventionally known coverlet material, such as polyurethane coated polyester, polyurethane coated nylon, or a combination of one or more layers of these materials.

The foam perimeter 20 defines an interior well 40 bounded by a floor 26, a top surface 28, and side and end walls 30, 32 which, in a preferred embodiment have interior facing surfaces 31, 33 diverging from each other as they extend upwardly from the floor 26 to the top surface 28. The foam perimeter 20 may be made of any conventionally known mattress bolster material, such as resilient polyurethane.

Referring also to FIG. 2, the actuator assemblies 12 comprise of a plurality of geometrically shaped, actuatable polymer actuators 34 that can expand and contract the actuators 34 per the patient's needs. The actuators 34 may also be heated or cooled via heating or cooling elements such as thermo voltaic chips 42 which also are controlled by the controller 16. For use in a hospital environment, the actuators 34 may be built bundled within a woven nylon fabric fused to a heavy gauge polymeric film. The actuators 34 may be used in place of the air cells of any conventional bed or pressure device such as that shown in U.S. Pat. No. 6,223,369, which is incorporated herein by reference. If desired, some of the actuators 34 may be replaced by foam elements 44. The actuators 34 and controller 16 preferably are as described in PCT/US04/05922 and PCT/US05/45210, and US 2007/73188, the contents of which are incorporated by reference, which as noted therein, include an electro-active polymer/electrolyte, working and connector electrodes, and an addressable controller, all as described in our aforesaid applications.

In use, the actuator and assembly 12 and its housing 20, and the foam perimeter, etc., nest closely within the confines of a hospital bed frame 50. The orientation of the actuator housing end walls 31, 33 may be angular to conform to the angular orientation of the bed frame side walls. This configuration of the end walls allows the actuators and their housing to extend nearly to the periphery of the support surface. This is very different from the prior art hospital support surfaces in which the mattresses are in-board of the bed frame side walls.

It should be understood that the actuator core may not extend along the entire longitudinal length of the support surface, for example, the foot section of the support surface may be comprised of a foam material, a plurality of different air cells, one or more actuators and housings or other conventionally known materials.

It should also be understood that the walls of the actuators and housings and perimeter are not rigid and are sealed and therefore the geometric terms used herein are intended to identify general shapes or generalized geometric appearance of objects. Hence, the geometric terms are not intended to be strictly defined in absolute terms, such as perfectly straight walls, surfaces and the like, due to inherent bowing of some materials and the rounding of corners. Similarly, the geometric terms are intended to represent three dimensional shapes in addition to two dimensional shapes, for example, the term trapezoidal also is intended to include an object's thickness rather than simply being planar.

It should also be understood that the number of actuators and housings or the size of each may differ without departure from the teachings herein. Furthermore, each may be comprised of several smaller cells. Also, pressure and/or temperature sensors 60 may be mounted on or within the system, and if desired, the actuators may be controlled in response to signals from the pressure or temperature sensors. The system also may include heating or cooling elements 62. And, the pressure sensors also may be programmed to monitor when a patient leaves a bed, and to sound an alarm.

The invention is susceptible to modification. For example, referring to FIG. 3, in like manner, the therapeutic pressure system of the present invention may be used, for example, as a therapeutic pressure boot or wrap 70, e.g., for wrapping a patient's legs or other extremity during or post-surgery. In the embodiment of a pressure sock, alternating pressure can be applied radially, and programmed to move vertically up the leg, starting at the lower calf and moving upward in sequence thereby facilitating blood flow deep within the patient to prevent not only problems that arise from deep vein thrombosis but other blood flow complications such as bed sores or ulcers caused by restricted blood flow at the capillary surface or skin level of the patient. During surgery and at times when the patient would need to be immobile for long periods, as when flying, driving long distances or sitting lying down for extended periods, such a device would keep blood moving and prevent DVT formation.

Yet other modifications are possible. For example, referring to FIG. 4, a pluarality of therapeutic pressure systems 70, similar to the therapeutic pressure systems above described, may be assembled together with the controller 72 of each system connected wirelessly or by wire to a central monitoring system or station 74 so that the individual therapeutic pressure systems and their associated patients may be monitored and controlled from a central location.

Although the invention has been shown and described in its preferred form, it should be understood that other modifications, additions or deletions may be made thereto without departure from the spirit and scope of the invention. For example, the system also may include conventional air bolsters, pumps and valves, e.g., for pumping conventional fluids to supplement the system.

What is claimed is:

1. A therapeutic pressure system, comprising: a sealed actuator housing containing an electrolyte, one or more polymer actuators that actuate via movement of electrolyte components into, out of, around or within the polymer matrix of the polymer actuators, an actuator support structure for the polymer actuators, working and counter electrodes in working contact with the actuators, circuitry for connecting the electrodes to a power source, and a controller between the power source and the electrodes to control the electrical current to the electrodes.

2. The therapeutic pressure system of claim 1 wherein the controller is programmable.

3. The therapeutic pressure system of claim 1 wherein the polymer actuators respond to or sense temperature.

4. The therapeutic pressure system of claim 1 wherein the polymer actuators respond to or sense pressure.

5. The therapeutic pressure system of claim 1 further including a heating element.

6. The therapeutic pressure system of claim 1 further including a temperature sensor or electronic thermometer.

7. The therapeutic pressure system of claim 1 further including a patient monitoring device.

8. The therapeutic pressure system of claim 1 further including a pressure sensor.

9. The therapeutic pressure system of claim 1 further including an auxiliary air bladder and valve.

10. The therapeutic pressure system of claim 1 further including an auxiliary air bladder and pump.

11. The therapeutic pressure system of claim 1 wherein the electrolyte is housed in a reservoir in fluid contact with the sealed actuator housing.

12. The therapeutic pressure system of claim 1 wherein the sealed actuator housing has fluid connection ports that remain closed unless a fluid conduit is connected to the fluid connection port.

13. The therapeutic pressure system of claim 1 further including an electrolyte heater.

14. The therapeutic pressure system of claim 1 comprising a plurality of therapeutic pressure system modular patient support assemblies.

15. The therapeutic pressure system of claim 14 wherein the controller is connected to the plurality of therapeutic pressure system modular patient support assemblies.

16. The therapeutic pressure system of claim 1 wherein the controller is connected to a combination of therapeutic pressure system modular patient support assemblies, and air bladder systems.

17. The therapeutic pressure system of claim 1 wherein the actuator support structure is flexible.

18. The therapeutic pressure system of claim 17 wherein the actuator support structure is less flexible than the polymer actuators.

19. The therapeutic pressure system of claim 1 shaped to cradle or surround a patient's extremity.

20. The therapeutic pressure system of claim 1 used as a mattress, portion of a mattress, covering of a mattress, built into a mattress or integrated mattress system for medical patients or therapeutic purposes in hospitals, patient care facilities and at home.

21. The therapeutic pressure system of claim 1 further including heating or cooling elements controlled by the controller.

22. The therapeutic pressure system of claim 1 shaped and sized to be used as a wheel chair cushion, seat, seat back or other surface in contact with a patient using a wheelchair or similar mobility device.

23. The therapeutic pressure system of claim 1 shaped and sized to be used as a pressure stocking or device to prevent or treat deep vein thrombosis and other venous disorders.

24. The therapeutic pressure system of claim 1 shaped and sized to be used as a stationary chair, part of a chair or incorporated into a chair.

25. The therapeutic pressure system of claim 24 wherein the chair or parts of the chair can be moved or are ‘positionable’ for increased patient comfort or therapeutic benefit.

26. The therapeutic pressure system of claim 1 further having a removable pad or covering.

27. The therapeutic pressure system comprising a plurality of therapeutic pressure systems of claim 1, wherein each controller is connected to a central monitoring system either wirelessly or by wire so that the therapeutic pressure systems and patients can be monitored through central monitoring system or station.
